(19) United States
(12) Patent Application Publication
(10) Pub. No.: US 2009/0048530 A1
(43) Pub. Date: Feb. 19, 2009

(54) MONITORING OF EPILEPTIFORM ACTIVITY

(75) Inventors: Mika Sarkela, Helsinki (FI); Bryan Young, London (CA)

Correspondence Address: ANDRUS, SCEALES, STARKE & SAWALL, LLP, 100 EAST WISCONSIN AVENUE, SUITE 1100, MILWAUKEE, WI, 53202, US

(73) Assignee: THE GENERAL ELECTRIC COMPANY, Schenectady, NY (US)

(21) Appl. No.: 11/839,009

(22) Filed: Aug. 15, 2007

(51) Int. Cl. A61B 5/0476

(52) U.S. Cl. 600/544

(57) ABSTRACT

The invention relates to monitoring of epileptiform activity. In order to accomplish a mechanism with improved specificity to epileptiform activity and with the capability to provide a clinician enough information for selecting a precision-targeted drug at an early phase of a seizure, first and second indicators are derived from the brain wave signal data obtained from a subject, the indicators being respectively indicative of the level of underlying neuronal excitation and neuronal inhibition. Based on the first and second indicators, an indication of the level of at least one of the neuronal excitation and neuronal inhibition is given to an end-user.

### FIELD OF THE INVENTION

The present invention relates generally to the monitoring of epileptiform activity. More particularly, the present invention relates to a mechanism for automatic detection and interpretation of epileptiform activity in subject's brain wave data. Epileptiform activity here refers to signal waveforms or patterns which are typical in epilepsy and encephalopathy, and which may also be associated with an increased risk of seizures.

### BACKGROUND OF THE INVENTION

Electroencephalography (EEG) is a well-established method for assessing brain activity. When measurement electrodes are attached on the skin of the skull surface, the weak biopotential signals generated in brain cortex may be recorded and analyzed. The EEG has been in wide use for decades in basic research of the neural systems of the brain as well as in the clinical diagnosis of various central nervous system diseases and disorders.

The EEG signal represents the sum of excitatory and inhibitory potentials of large numbers of cortical pyramidal neurons, which are organized in columns. Each EEG electrode senses the average activity of several thousands of cortical pyramidal neurons.

The EEG signal is often divided into four different frequency bands: Delta (0.5-3.5 Hz), Theta (3.5-7.0 Hz), Alpha (7.0-13.0 Hz), and Beta (13.0-32.0 Hz). In an adult, Alpha waves are found during periods of wakefulness, and they may disappear entirely during sleep. Beta waves are recorded during periods of intense activation of the central nervous system. The lower frequency Theta and Delta waves reflect drowsiness and periods of deep sleep.

Different derangements of internal system homeostasis disturb the environment in which the brain operates, and therefore the function of the brain and the resulting EEG are disturbed. The EEG signal is a very sensitive measure of these neuronal derangements, which may reflect in the EEG signal either as changes in membrane potentials or as changes in synaptic transmission. A change in synaptic transmission occurs whenever there is an imbalance between consumption and supply of energy in the brain. This means that the EEG signal serves as an early warning of a developing injury in the brain.

According to the present state of knowledge, the EEG signal is regarded as an effective tool for monitoring changes in the cerebral state of a patient. Diagnostically, the EEG is not specific, since many systemic disorders of the brain produce similar EEG manifestations. For example, in Intensive Care Units (ICU), an EEG signal is of critical value, as it may differentiate between broad categories of psychogenic, epileptic, metabolic-toxic, encephalopatic and focal conditions.

Epilepsy is the most common chronic neurological disorder, affecting about one percent of the population at some time in their life. Epileptic seizure activity is experienced by about 8% of the patients in general ICU environment where it is associated with increased morbidity and mortality. In particular categories, such as in coma patients, children, patients with prior clinical seizures, central nervous system infections, head trauma, brain tumor, or recent neurosurgery, the risk of seizures is even higher.

From clinical observations it is known that many different types of epileptic seizures and many epileptic syndromes do not share a common pathogenesis. Studies conducted with patients of temporal lobe epilepsy associated with hippocampal sclerosis indicate that epileptogenesis is iniated by specific types of cell loss and neuronal reorganization, including increased density of excitatory synapses, enhanced release of excitatory neurotransmitters, and functional or anatomical loss of inhibitory influences. This process results in enhanced neuronal excitability and/or altered neuronal inhibition, predisposing to neuronal hypersynchronization in the particular brain area. Enhanced neuronal excitability refers to a process where action potentials are more likely to occur, i.e. voltage over the (brain) cell membrane is above the usual value of about −60 mV, in other words the cell membrane is depolarized. Altered neuronal inhibition refers to a process where action potentials are less likely to occur, i.e. voltage over the cell membrane is below the usual value of −60 mV, in other words the cell membrane is hyperpolarized. Neuronal hypersynchronization refers to a process where multiple neurons are affected by simultaneous action potentials. Hypersynchronization has been verified by intracranial EEG recordings, cf. McSharry et al.: Comparison of Predictability of Epileptic Seizures by a Linear and Nonlinear Method, IEEE Transactions on Biomedical Engineering, vol. 50, No. 5, May 2003, pp. 628-633, which shows a reduction of EEG signal complexity in the area of seizure focus. However, when brain activity is recorded from the scalp, the measured signal is a composition originating from multiple sources and methods indicative of the complexity of the signal may show an increase during a seizure, cf. U.S. Pat. Nos. 5,743,860 and 5,857,978.

Just as there are numerous seizure types, any type of seizure may manifest as status epilepticus (SE). SE is usually defined as more than 30 minutes of (1) continuous seizure activity, or (2) two or more sequential seizures without full recovery of consciousness between the seizures. Status epilepticus is often divided into convulsive and non-convulsive types. The majority of the seizures in ICU are non-convulsive and occur in comatose patients, so they can only be detected by EEG. The EEG, which demonstrates ongoing ictal activity, can be used to further subdivide SE into either generalized (abnormal activity in the whole brain) or partial SE (abnormal activity in a particular region of the brain). Convulsive status epilepticus (CSE) is the most serious, frequent, and most easily recognized type of SE. It may occur in primary generalized epilepsy or be secondarily generalized. CSE is characterized by loss of consciousness and recurrent or continuous convulsions. Non-convulsive status epilepticus is often defined as an epileptic state of more than 30 minutes with some clinically evident change in mental status or behaviour from baseline (again this may not be obvious in the already comatose ICU patient) and ictal activity in the EEG. Until recently, neural damage was only associated with prolonged seizures as with SE. However, emerging experimental studies in chronic models, human magnetic resonance imaging, and neuropsychological studies have provided evidence that even single seizures and repeated brief seizures can produce neuronal damage and death. Seizure-induced cell death and damage may also adversely affect the functional properties of neural circuits and networks, and subtle seizure-induced neuronal loss or circuit reorganization may have clinically significant impacts on cognition and behavior.

Although a single epileptiform spike lasts only for a fraction of a second, long-term EEG monitoring can reflect slow trend changes. If a seizure occurs during the monitoring period, the EEG signal can be used to categorize the epileptiform patterns and seizure activity as a specific epilepsy classification, including the non-convulsive forms of status epilepticus. In addition, the EEG signal may be used as a control tool for inducing anesthesia to a level where there are no recognizable seizures, without producing excessive suppression of neural activity. Encephalopathy commonly refers to central nervous system dysfunction of any cause, and it can be classified further as either an epileptic encephalopathy or epileptiform encephalopathy. While epileptic encephalopathies are characterized by frequent seizures, epileptiform encephalopathies refer to disorders with epileptiform activity without marked clinical seizure activity. As mentioned above, epileptiform activity refers in this context generally to signal waveforms or patterns that are typical in epilepsy and its associated encephalopathy, and patterns associated with an increased risk of seizures.

Most of the metabolic and systemic disorders have EEG correlates, and if there is a disturbance of conscious level, the EEG is never normal. Due to the close relationship between epilepsy and encephalopathy, similar waveforms or patterns may also appear in other states than epilepsy, such as in metabolic encephalopathy. It is also to be noted in this context that detected epileptiform activity does not alone confirm a diagnosis, but the patient needs to be further examined. However, the EEG findings in encephalopathy have many similarities to those during sedation and anesthesia, which makes the detection of encephalopathy in sedated patients difficult. Generally, when a patient loses consciousness, a shift of spectral power towards lower frequencies appears. Generalized slowing applies also in the case of epileptiform encephalopathy, however additive periodical and miscellaneous patterns often appear in the EEG. Periodic patterns can be, for example, periodic lateralizing epileptiform discharges (PLEDs), generalized periodic epileptiform discharges (GPEDs) or burst-suppression. Miscellaneous patterns may be, for example, triphasic waves, which occur in about 20-25% of the hepatic encephalopathy patients. However, triphasic waves are not specific for this disease, but may also occur in other metabolic diseases. In addition to low frequency activity, epileptiform activity may include spiky waveforms, reaching up to about 70 Hz on the spectral range.

Numerous automatic techniques have been described for the detection and prediction of epileptiform activity. Most of the known methods utilize the EEG signal from wide frequency range, for example 1-32 Hz. Therefore, the methods are not specific enough to epileptiform activity only but are also sensitive to the dynamical EEG changes occurring during sedation, surgical anesthesia or normal wake-sleep cycle. For example, a spectral entropy has been utilized for investigating the relationships between epileptiform discharges and background EEG activity, cf. T. Inouye et al.: Abnormality of background EEG determined by the entropy of power spectra in epileptic patients, Electroencephalography and clinical Neurophysiology, 82 (1992), pp. 203-207. The above-mentioned U.S. Pat. Nos. 5,743,860 and 5,857,978 in turn describe analysis methods in which the detection of epilectic seizures is based on non-linear measures of the signal data, such as Kolmogorov entropy. The signal data may be EEG signal data or magnetoencephalographic (MEG) signal data. MEG is indicative of the magnetic component of brain activity, i.e. it is the magnetic counterpart of EEG. However, entropy is also used for the purposes of monitoring depth of anesthesia in surgical patients, cf. U.S. Pat. No. 6,731,975. Because entropy variables derived from a wide frequency-band EEG signal are sensitive to drug-induced anesthesia, natural sleep and epileptiform activity, the applicability of these methods for the monitoring of encephalopatic patients is naturally limited.

Methods based on wavelet transformation of the EEG signal data have also been proposed for analyzing brain signals, cf. Rosso O A, Blanco S, Yordanova J, Kolev V, Figliola A, Schurmann M, Basar E: Wavelet entropy: a new tool for analysis of short duration brain electrical signals. Journal of Neuroscience Methods 105 (2001), pp. 65-75. In this particular method, entropy is calculated from the power distribution between the decomposition levels of the transform. In that sense, the technique is thus related to the determination of spectral entropy. However, spectral information is now derived by means of a wavelet transform instead of a Fourier transform.

The article Rosso O A, Blanco S., Rabinowitz A. Wavelet analysis of generalized tonic-clonic epileptic seizures, Signal Processing 2003; 83(6): 1275-1289, describes a wavelet-based method for the analysis of generalized tonic-clonic epileptic seizures. The identification of these seizures is aggravated by the simultaneous muscle activity disturbing the EEG signal. The article describes that wavelet entropy corresponding to a frequency band of 0.8 to 12.8 Hz is lower during seizures than during pre- and post-seizure periods. When a wider frequency band of 0.8 to 51.2 Hz is used, the wavelet entropy first increases at the beginning of a seizure, which may be caused by muscle activity.

A further wavelet-based method for analyzing an EEG is described in Geva A B, Kerem D H: Forecasting Generalized Epileptic Seizures from EEG Signal by Wavelet Analysis and Dynamic Unsupervised Fuzzy Clustering, IEEE Transactions on Biomedical Engineering, vol. 45, October 1998, pp. 1205-1216. The method, which is intended for forecasting a generalized epileptic seizure, relies on the availability of the EEG signal preceding the seizure and utilizes fuzzy clustering for classifying temporal EEG patterns.

One drawback related to the above techniques for automatic detection of epileptiform activity is the weak specificity for epileptiform activity, which is manifested as high false positive detections. The above techniques cannot distinguish epileptiform activity from brain wave activity changes caused by the variations in the level of consciousness of the patient. For example, in the above-described methods based on wavelet entropy the entropy values obtained during an epileptic seizure are typically between the wavelet entropies of the conscious and unconscious states of a patient. Therefore, the methods cannot distinguish, for example, whether an increase in the wavelet entropy is caused by an epileptiform EEG of anesthetized patient or the arousal of the patient.

A further drawback of the prior art detection techniques is that they cannot indicate when a specific type of epileptiform activity is present in the EEG or which type of epileptiform waveforms are present in the EEG signal. Consequently, prior art detection techniques cannot provide a clinician with enough information for selecting a precision-targeted drug. Rather, the clinician is bound to select an antiepilectic drug (AED) somewhat arbitrarily, since (s) he has no knowledge of the specific nature of the epileptiform activity.

The present invention seeks to eliminate the above-mentioned drawbacks and to bring about a mechanism for detecting epileptiform activity with improved specificity and with the capability to provide enough information about the nature of epileptic activity for proper selection of drugs.

### SUMMARY OF THE INVENTION

The present invention seeks to provide a novel mechanism that enables automatic and reliable detection of epileptiform activity in brain wave signal data, regardless of possible changes in patient's level of consciousness. The present invention further seeks to provide a mechanism that provides information about the neuronal mechanisms related to epileptiform activity, to enable timely treatment of the patient with precision-targeted drugs.

The present invention rests on the theory that epileptiform activity can generally be understood as an imbalance between two opposite mechanisms of neurotransmission: excitatory and inhibitory. Neuronal excitation is mediated by excitatory neurotransmitters, including glutamate, aspartate and acetylcholine, whereas neuronal inhibition is mediated chiefly by gamma amino butyric acid (GABA), which acts on GABA-A receptors to open the chloride ionophore, causing the neuron to become hyperpolarized and less excitable. Antiepileptic drugs (AEDs) generally affect either by depressing excitation or by increasing inhibition. For example, propofol acts via the GABA-A receptor system increasing the inhibitory property of the system. Barbiturates, e.g. phenobarbital, increase chloride conductance, causing neuronal hyperpolarization. Benzodiazepines, such as diazepam, lorazepam and midazolam, act on the benzodiazepine receptor that is also linked to the chloride ionophore, and enhance inhibition. Ketamine is the only commonly-available agent that directly acts to inhibit the N-methyl-D-aspartate (NMDA) receptor (which opens a channel for calcium and sodium influx), but a number of other drugs, e.g. phenytoin, carbamazepine and valproate, reduce excitation by decreasing the conductance of rapidly acting sodium channels.

In the present invention, indicators are derived from the brain wave signal data, which describe the degree of the above-described neuronal mechanisms of epileptiform activity. At least two indicators are therefore derived: one indicative of the degree of excitation and another indicative of the degree of inhibition. An indication of the level of at least one of the neuronal excitation and neuronal inhibition is then given to the end-user. This may be given continuously, but at least when an imbalance is detected, i.e. when either of the mechanisms becomes dominant. The current levels of the two neuronal mechanisms may be indicated on a continuous scale or as relative levels compared to the normal value of the respective mechanism or to the level of the opposite mechanism, for example.

Thus one aspect of the invention is providing a method for monitoring epileptiform activity. The method comprises deriving a first indicator from brain wave signal data obtained from a subject, the first indicator being indicative of the level of neuronal excitation. The method also comprises deriving a second indicator from the brain wave signal data, the second indicator being indicative of the level of neuronal inhibition, and giving, based on the first and second indicators, an indication of the level of at least one of the neuronal excitation and neuronal inhibition.

Excitatory mechanisms are characteristically observed at an EEG frequency range of approximately 16 Hz and above, and inhibitory mechanism at a frequency range of approximately below 16 Hz. One suitable technique for detecting EEG activity on these frequency bands is a wavelet-based technique, due to its orthogonal property to decompose the measured signal to different frequency bands.

With the help of the information provided by the invention, it is possible to medicate patients by a precision-targeted drug that either decreases excitation or increases inhibition. Consequently, one aspect of the invention is the provision of a method for the selection of an anti-epileptic drug for a subject with detected epileptiform activity.

A further advantage of the invention is that it provides an early warning of an incipient seizure, thereby enabling an early meditation with a precision-targeted drug, which is important for minimizing the duration of the seizure and the brain damage caused by the seizure.

In one embodiment of the invention, the two indicators may be used to derive a third indicator, directly indicative of the balance between excitatory and inhibitory mechanisms and thus also of the currently dominant mechanism of neurotransmission. The third indicator may be displayed on a continuous scale, for example, to indicate the degree of dominance of one of the mechanisms. However, various alternatives may be used to present the degree and type of the underlying neuronal activity in an excitation-inhibition space.

Another aspect of the invention is that of providing an apparatus for detecting epileptiform activity. The apparatus comprises a first calculation unit configured to derive a first indicator from brain wave signal data obtained from a subject, the first indicator being indicative of the level of neuronal excitation, and a second calculation unit configured to derive a second indicator from the brain wave signal data, the second indicator being indicative of the level of neuronal inhibition. The apparatus also comprises an indicator unit configured to give, based on the first and second indicators, an indication of the level of at least one of the neuronal excitation and neuronal inhibition.

In a still further embodiment, the invention provides a computer program comprising computer program code means adapted to perform the above steps of the method when run on a computer. It is, however, to be noted that since a conventional EEG/MEG measurement device may be upgraded by a plug-in unit that includes software enabling the measurement device to detect the relative levels of excitation and inhibition, the plug-in unit does not necessarily have to take part in the acquisition of the brain wave signal data.

Other features and advantages of the invention will become apparent by reference to the following detailed description and accompanying drawings.

### BRIEF DESCRIPTION OF THE DRAWINGS

In the following, the invention and its preferred embodiments are described more closely with reference to the examples shown in FIG. 1 to 7 in the appended drawings, wherein:

FIG. 1 illustrates the basic steps of the method of the invention;

FIG. 2 illustrates an embodiment of the invention, in which a wavelet transform is utilized to detect neuronal excitation and inhibition;

FIG. 3 illustrates an example of the embodiment of FIG. 2;

FIG. 4 illustrates the subband coding performed in the embodiments of FIGS. 2 and 3;

FIG. 5 illustrates the detection of epileptiform activity by the indicators of the present invention;

FIG. 6 illustrates one embodiment of the apparatus according to the invention; and

FIG. 7 illustrates the operational units of the apparatus of FIG. 6 for detecting epileptiform activity in the EEG signal data. FIG. 1

### DETAILED DESCRIPTION OF THE INVENTION

Below, different embodiments of the invention are discussed assuming that the brain wave signal data measured from the patient is EEG signal data.

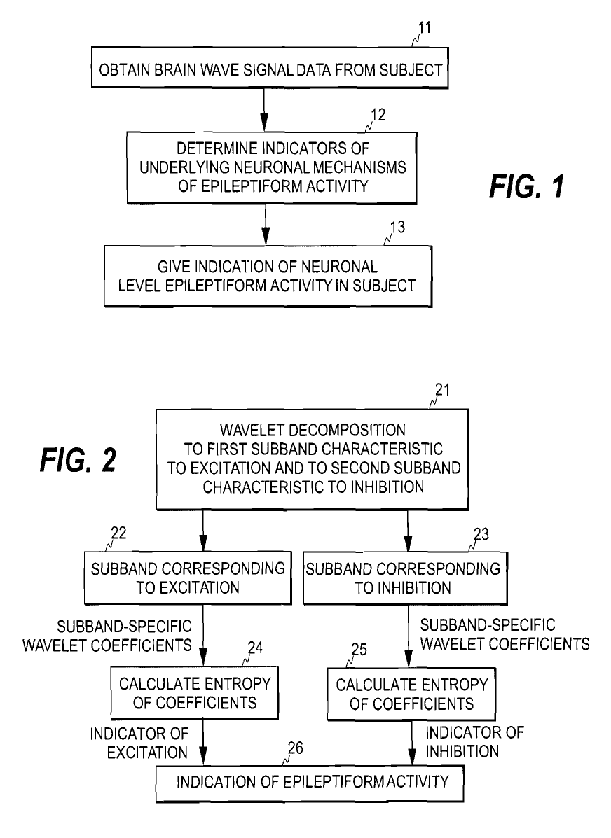

FIG. 1 is a flow diagram illustrating the detection mechanism of the invention. Based on the brain wave signal data obtained from a subject at step 11, a set of indicators are derived from the brain wave signal data for each time window of the signal data (step 12). The set includes indicators indicative of the degree of the two opposite neuronal level mechanisms of epileptiform activity: a first indicator indicative of the degree of excitation and a second indicator indicative of the degree of inhibition. Based on the indicators, an indication is given to the end-user of the level of at least one of the neuronal mechanisms in the subject (step 13). As discussed below, this may be carried out in various ways depending on how the above two indicators are employed to produce the information supplied to the end-user.

In case of epileptiform activity, increased excitation is typically observed as an increase of spike activity of EEG. Spike here refers to sharp transients of a duration up to about 200 ms. Inhibition is in turn related to slow wave activity of the EEG. Increasing level of inhibition will lead to lower frequencies of EEG waveform until total suppression is reached. During a single epileptic seizure evolutional changes of the EEG patterns can be observed, cf. Blume W T, Young G B, Lemieux J F: EEG morphology of partial epileptic seizures, Electroenceph. Clin. Neurophysiol. 1984, 57; 295-302. These patterns correspond to the excitation/inhibition balance of that moment. Seizures can start, for example, with a monotonic increase of EEG spike amplitudes, reflecting a continuous increase in the overshoot of excitation. After the peak point of excitation is reached, inhibitory mechanism starts and further evolves towards lower frequencies until suppression is reached. Different evolutional forms of seizures also exist, and some seizures may contain only inhibitory or excitatory activity.

Excitatory mechanisms are characteristically observed at an EEG frequency range of about 16 Hz to up to even 1 kHz and inhibitory mechanism at a frequency range of about 0 to about 16 Hz. Although different mechanisms may be utilized to calculate indicators indicative of the level of EEG activity on these frequency bands, one suitable technique for detecting activity on these frequency bands is a wavelet based technique, due to its orthogonal property to decompose the measured signal to different frequency bands. In this technique, the entropy of the wavelet coefficients of the frequency bands that correspond to excitation and inhibition indicates the degree of the respective underlying neuronal mechanism of epileptiform activity. The entropy of the wavelet coefficients obtained from a subband of the brain wave signal data is in this context termed wavelet subband entropy (WSE). Subband entropy decreases during epileptiform activity, i.e. decreasing WSE is a sign of increasing underlying neuronal activity of a specific type (excitation or inhibition).

FIG. 2 illustrates the use of WSE for detecting the levels of excitation and inhibition. A wavelet-based filter bank, i.e. a filter bank configured to perform a wavelet transform, may be employed to decompose the EEG signal into subbands that are characteristic to excitation and inhibition (step 21). As a result of the decomposition, two sets of wavelet coefficients are obtained: a first set of coefficients corresponding to the subband of excitation (step 22) and a second set of coefficients corresponding to the subband of inhibition (step 23). As is common in the art, the digitized signal samples are processed as sets of sequential signal samples representing finite time blocks or time windows, commonly termed “epochs”. Therefore, the coefficient sets are obtained for each epoch.

The entropy of the wavelet coefficients of each subband is then calculated at steps 24 and 25 for each epoch to determine the degree of the respective underlying neuronal mechanism of epileptiform activity. Step 24 thus yields a time series of a first indicator indicative the degree of excitation, while step 25 yields a time series of a second indicator indicative the degree of inhibition, the indicators being in this example the entropies computed over the coefficients of a particular level of the wavelet transformation.

The two indicators are then utilized at step 26 to provide the end-user with information about the current degree of the underlying neuronal mechanisms of epileptiform activity in the patient. In one embodiment, the apparatus of the invention may simply display each of the indicators on a continuous scale to give the end-user a notion of the levels of the opposite neuronal mechanisms. In another embodiment, an indicator of the balance between excitatory and inhibitory mechanisms may be determined based on the two indicators. This indicator may be, for example, the ratio of the two indicators. The ratio may be presented on a continuous scale. However, various presentation mechanisms may be employed to give the end-user an idea of the relative levels of neuronal excitation and inhibition. For example, the status of the patient may be shown on an excitation/inhibition plot or coordinate system.

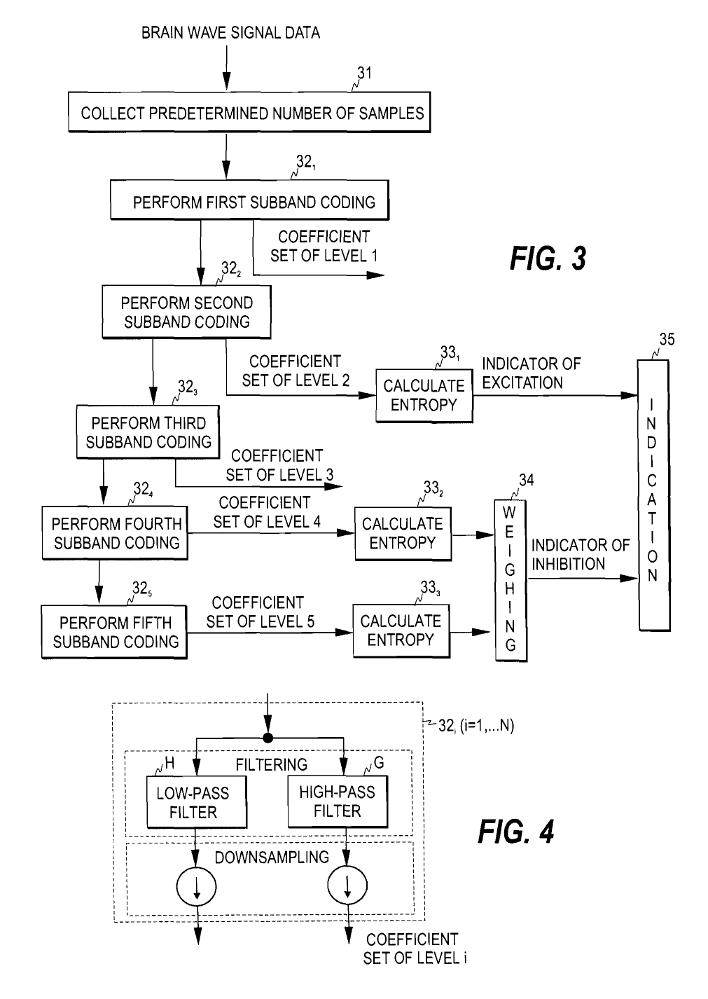

FIG. 3 illustrates an example of the embodiment of FIG. 2. The incoming EEG signal is sampled at a predetermined sampling frequency and therefore the process first collects a predetermined number of samples representing the signal in a time window of a predetermined length (step 31). Each epoch is supplied to a subband coding process, which is performed five times (steps 32₁ to 32₅) in this example. Subband coding here refers to the filtering and downsampling operations performed at each decomposition level of a discrete wavelet transform. FIG. 4 illustrates the said operations. As common in discrete wavelet transforms, at the first decomposition level the original signal is first passed through a high-pass filter G and a low-pass filter H. After the filtering, part of the samples, typically half, is discarded in a downsampling process. The output of the high-pass filter constitutes the wavelet coefficients of the respective decomposition level, namely approximation coefficients characterizing the signal on a coarse scale and detail coefficients characterizing the signal on a fine scale. At the successive decomposition levels, the approximation coefficients are passed through identical filters followed by downsampling. The number of coefficients obtained depends on various parameters, such as the decomposition level in question.

With reference back to FIG. 3, the subband coding is thus in this case performed five times, each subband coding corresponding to a certain decomposition level. The number of the subband coding processes to be carried out depends on the subbands of interest and on the sampling frequency; the subband coding is repeated until each subband of interest is available.

For reducing computational load of the method, it is advantageous to use relatively low sampling frequencies when measuring the EEG signal. Since the frequency band, which covers both types of the epileptiform activity extends approximately from 0 Hz to 70 Hz, the sampling frequency may be, for example, 128 Hz. As the scales of the wavelet transform have a rough analogy to frequency space, a frequency band convention is used henceforth instead of scales. With the above choice of the sampling frequency, the detail coefficients of the first decomposition level correspond to a subband of 32 to 64 Hz (if dyadic sampling is used), the detail coefficients of the second level to a subband of 16 to 32 Hz, the detail coefficients of the third level to a subband of 8 to 16 Hz, the detail coefficients of the fourth level to a subband of 4 to 8 Hz, and the detail coefficients of the fifth level to a subband of 2 to 4 Hz. By performing five successive subband coding processes the coefficients are thus obtained for the subbands corresponding to excitation and inhibition. In this example, the level of excitation is evaluated by computing the entropy of the detail coefficients obtained from the subband of 16 to 32 Hz and the level of inhibition by computing the WSE of the subband of 2 to 8 Hz. Therefore, an indicator of excitation is obtained by calculating the entropy of the detail coefficients of the second decomposition level (step 33₁). An indicator of inhibition is in turn obtained by first calculating the entropies of the detail coefficients of the fourth decomposition level corresponding to the subband of 4 to 8 Hz (step 33₂) and the fifth decomposition level (step 33₃) corresponding to the subband of 2 to 4 Hz, and then determining a weighed average of the two entropies (step 34) to obtain the WSE of the subband of 2 to 8 Hz. The indicator of excitation, obtained from step 33₁, and the indicator of inhibition, obtained from step 34, are then utilized to provide the end-user with information about the levels of the two opposite neuronal mechanisms in the subject (step 35).

The detection of specific epileptiform waveforms is discussed in European Patent Application EP 06110089.7 and on U.S. patent application Ser. No. 11/617,151 of the Applicant, the contents of which are incorporated herein by reference. The said applications disclose different mathematical methods possible for obtaining an indicator indicative of the presence of epileptiform activity of a specific type. One of the methods disclosed utilizes the above WSE as the said indicator. However, as is discussed in the said applications, kurtosis, which is a normalized form of the fourth central moment, may be used instead of entropy to indicate the presence of a specific type of epileptiform waveforms. Furthermore, kurtosis may be replaced by a normalized form of a central moment of an order higher than four. The methods disclosed in the said applications may be used to calculate the two indicators of the present invention, if the same mathematical methods are applied to subbands of excitation and inhibition and if measures of the activity levels of the two subbands are determined.

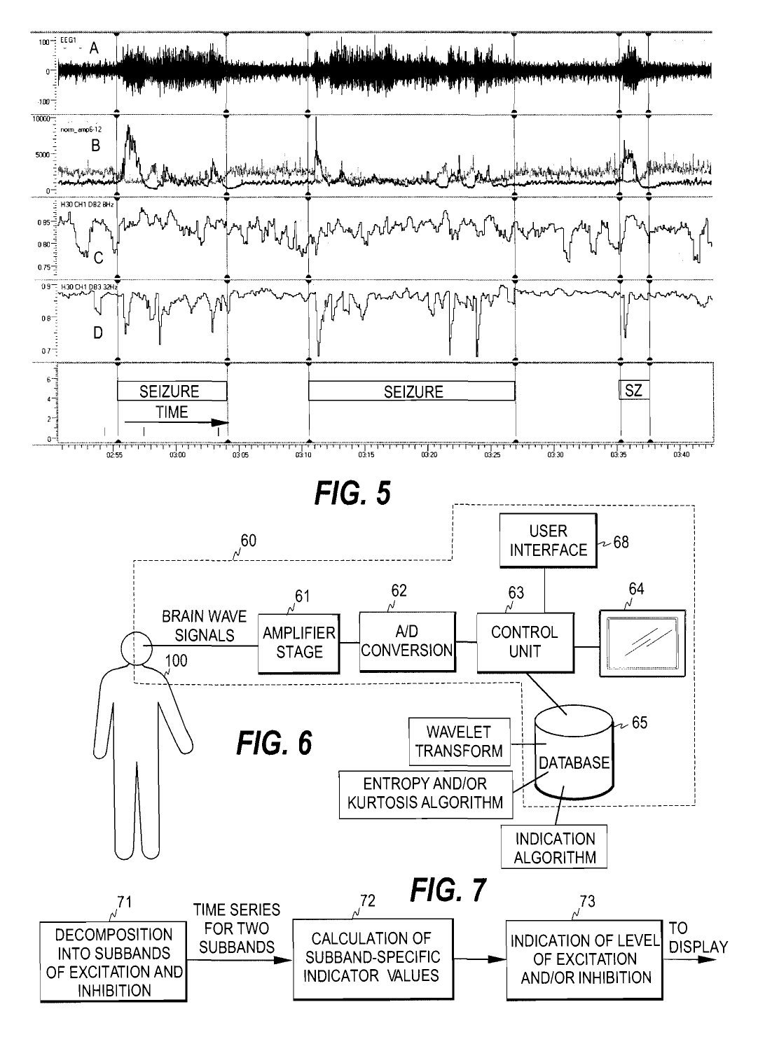

FIG. 5 illustrates the detection of excitation and inhibition by showing a one-hour recording of an ICU patient with three non-convulsive seizures marked offline by a neurophysiologist. Line A shows the actual EEG signal. Line B shows two essential features, coefficient of variation (grey) and relative amplitude (black), used in a known seizure detection algorithm (Y. U. Khan & J. Gotman: Wavelet based automatic seizure detection in intracerebral electroencephalogram, Clinical Neurophysiology, vol. 114, 2005, pp. 898-908). Lines C and D show, respectively, the indicators of inhibition and excitation. The indicator of excitation is in this example calculated according to the embodiment of FIG. 3, while the indicator of inhibition is calculated as the WSE of the subband of 4 to 8 Hz, i.e. In this example the output of step 33₂ in FIG. 3 alone represents the indicator of inhibition. As can be seen from the figure, low subband entropy, indicative of increased inhibition, precedes each marked seizure. Therefore, the indicator of inhibition may provide an early warning of a developing seizure. Furthermore, increased excitation during the marked seizures is captured by low values of the wavelet entropy of the subband of 16 to 32 Hz. It should be noted that enhanced inhibition preceding each marked seizure may actually represent the seizure itself, since seizures may start with either dominant inhibition or dominant excitation, and turn to the dominance of the other mechanism during the seizure. As can be seen from the figure, the method is transcendent over the state-of-the-art method of line B in this respect, since the state-of-the-art method only indicates the excitatory periods.

The efficiency of the invention in detecting an incipient seizure may be employed in administering a precision-targeted drug to the patient automatically or by guiding the clinical personnel to select an appropriate drug. In this example, an anti-epileptic drug enhancing inhibition, e.g. propofol, diazepam, lorazepam or midazolam, could be administered to the patient during the periods of enhanced inhibition prior to each marked seizure. As FIG. 3 proposes, the method of the invention may even recognize specific types of epileptiform activity, which may remain undetected by a medical specialist, and more importantly, the invention is able to provide real-time information about the prevailing neuronal mechanisms on the bed space.

In the above examples, the brain wave signal data obtained from a subject is decomposed to obtain subband-specific output data for the subbands on which excitation and inhibition typically appear. The output data presents a time series of a quantitative characteristic of the brain wave signal data on these subbands. In the above examples, the wavelet coefficients form the quantitative characteristic and the level of excitation/inhibition is evaluated by calculating the WSE from the subbands corresponding to excitation and inhibition. The advantage of the WSE is its specificity to different waveforms, especially to those appearing during high levels of excitation/inhibition, which are typical in epileptiform brain activity and do not normally appear in anesthesia. However, other indicators of the level of excitation and inhibition may also be used. In addition to WSE, possible indicators of the level of inhibition include EEG signal power on Delta and/or Theta bands, spectral entropy calculated over a wide EEG band, and burst suppression ratio (BSR), for example. Since the peakedness of the EEG signal increases as excitation increases, indicators of EEG peak amplitude or EEG peak rate are also possible indicators of the level of excitation. Furthermore, EEG power on a higher frequency band may be used as an indicator of the level of excitation.

In one WSE-based embodiment of the invention, different mother wavelets are used for the subbands of inhibition and excitation. For example, it has been discovered that Daubechies 1 (db1) and Daubechies 2 (db2) basis function efficiently captures inhibition, while Daubechies 3 (db3) basis function works well in case of excitation.

FIG. 6 illustrates one embodiment of the system according to the invention. As mentioned above, the brain wave data acquired from a patient is typically EEG signal data. The EEG signal is typically measured from the forehead of the patient 100, which is a preferred measurement site due to the ease of use of the measurement and the reduced inconvenience caused to the patient.

The signals obtained from the EEG sensors are supplied to an amplifier stage 61, which amplifies the signals before they are sampled and converted into digitized format in an A/D converter 62. The digitized signals are then supplied to a control unit 63 (including a microprocessor), which may then record the signals as an EEG time series.

The control unit is provided with a database or memory unit 65 holding the digitized EEG signal data obtained from the sensors. Before the actual detection algorithm, the control unit may perform various pre-processing phases for improving the quality of the EEG signal data or the said phases may be carried out in separate elements between EEG sensors and the control unit. The actual recording of the EEG signal data thus occurs in a conventional manner, i.e. the measurement device 60 including the above elements serves as a conventional EEG measurement device. However, certain parameters, such the sampling frequency of the device, may be set according to the requirements of the decomposition process so that the separated frequency bands correspond to neuronal excitation and inhibition.

Additionally, the control unit is provided with the above-described algorithms for detecting epileptiform waveforms in the EEG signal data. As shown in FIG. 7, the control unit may thus include three successive operational entities: a first entity 71 for decomposing the EEG signal data in order to obtain the output data (time series) for the subbands of excitation and inhibition, a second entity 72 for calculating the excitation and inhibition indicator values, such as WSE values, based on the time series, and a third entity 73 for giving an indication of the level of excitation and/or inhibition. As discussed above, the third entity may determine one or more parameters indicative of the imbalance between excitation and inhibition and may present the results in various ways in an excitation-inhibition space.

The first entity typically includes a wavelet-based filter bank yielding a time series of wavelet coefficients, but may also include at least one filter yielding a time series of signal amplitude or signal power for the subbands of excitation and inhibition. In a simplified embodiment of the invention, the third entity may also be an indicator module that presents the indicator values to the user so that the user may deduce the relative levels of excitation and inhibition.

Although one control unit (data processing entity) may perform the calculations needed, the processing of the EEG signal data obtained may also be distributed among different data processing entities within a network, such as a hospital LAN (local area network). For example, a conventional measurement device may record the EEG signal data and an external computing entity, such as processor or server, may be responsible for determining the indicators of excitation and inhibition.

The control unit may display the results on the screen of a monitor 64 connected to the control unit. This may be carried out in many ways using textual and/or graphical information about the current levels of the underlying neuronal mechanisms of epileptiform activity. The information displayed may also comprise the normal levels of excitation and inhibition, in order that the end-user may compare the current level to the normal level. For example, if WSE is used as a monitoring parameter normal levels are approximately at 0.8 and above, whereas abnormal levels are all below about 0.8.

The system further includes user interface means 68 through which the user may control the operation of the system.

As discussed above, the brain wave data may also be acquired through a standard MEG recording. The measurement device 60 may thus also serve as a conventional MEG measurement device, although a MEG measuring arrangement is far more expensive than an EEG measuring arrangement. The software enabling a conventional EEG or MEG measurement device 60 to detect epileptiform waveforms may also be delivered separately to the measurement device, for example on a data carrier, such as a CD or a memory card, or through a telecommunications network. In other words, a conventional EEG or MEG measurement device may be upgraded by a plug-in unit that includes software enabling the measurement device to determine the relative levels of excitation and inhibition based on the signal data it has obtained from the patient.

Since the algorithm for detecting the waveforms does not require high computation power, it may also be used in various ambulatory devices, such as portable patient monitors, for monitoring epileptiform waveforms. The algorithm may also be introduced into various devices operating outside a clinical environment, such as mobile phones, PDA devices, or vehicle computers, which allows the monitoring of possible epileptic symptoms during day-to-day activities. However, the invention is most useful at bed space in enabling a clinician to choose a precision-targeted drug at an early stage of a seizure, thereby minimizing the adverse effects of the seizure.

Although the invention was described above with reference to the examples shown in the appended drawings, it is obvious that the invention is not limited to these, but may be modified by those skilled in the art without departing from the scope and spirit of the invention. For example, the limits of the excitation and inhibition subbands may vary and in wavelet-based embodiments continuous wavelet transform, discrete wavelet transform, or wavelet packet transform may be used to decompose the brain wave signal.

We claim:

1. A method for monitoring epileptiform activity, the method comprising:
deriving a first indicator from brain wave signal data obtained from a subject, the first indicator being indicative of the level of neuronal excitation;
deriving a second indicator from the brain wave signal data, the second indicator being indicative of the level of neuronal inhibition; and
giving, based on the first and second indicators, an indication of the level of at least one of the neuronal excitation and neuronal inhibition.

2. A method according to claim 1, wherein the giving the indication comprises displaying the values of the first and second indicators on respective continuous scales, wherein the displaying is performed substantially continuously.

3. A method according to claim 1, wherein the giving the indication comprises deriving a third indicator from the first and second indicators, the third indicator being indicative of the balance between the neuronal excitation and neuronal inhibition.

4. A method according to claim 3, wherein the giving the indication further comprises displaying the third indicator on a continuous scale.

5. A method according to claim 1, wherein
the deriving the first indicator comprises (i) decomposing the brain wave signal data into a first subband indicative of the neuronal excitation, to obtain first output data representing a time series of a quantitative characteristic of the brain wave signal data on the first subband and (ii) determining the first indicator as one of a first measure indicative of the entropy of the first output data and a second measure indicative of a normalized form of k:th order central moment of the first output data; and
the deriving the second indicator comprises (i) decomposing the brain wave signal data into a second subband indicative of the neuronal inhibition, to obtain second output data representing a time series of a quantitative characteristic of the brain wave signal data on the second subband and (ii) determining the second indicator as one of a third measure indicative of the entropy of the second output data and a fourth measure indicative of a normalized form of k:th order central moment of the second output data,
wherein k is an integer higher than three.

6. A method according to claim 5, wherein the determining includes determining the first indicator and the second indicator, in which the first indicator is indicative of the entropy of the first output data and the second indicator is indicative of the entropy of the second output data, wherein the first and second output data comprise wavelet coefficients.

7. A method according to claim 6, wherein the decomposing comprises decomposing the brain wave signal data into the first and second subbands, in which the first subband is substantially in its entirety above 16 Hz and the second subband is substantially in its entirety below 16 Hz.

8. An apparatus for monitoring epileptiform activity, the apparatus comprising:
a first calculation unit configured to derive a first indicator from brain wave signal data obtained from a subject, the first indicator being indicative of the level of neuronal excitation;
a second calculation unit configured to derive a second indicator from the brain wave signal data, the second indicator being indicative of the level of neuronal inhibition; and
an indicator unit configured to give, based on the first and second indicators, an indication of the level of at least one of the neuronal excitation and neuronal inhibition.

9. An apparatus according to claim 8, wherein the indicator unit is configured to display the values of the first and second indicators on respective continuous scales.

10. An apparatus according to claim 8, wherein the indicator unit is configured to derive a third indicator from the first and second indicators, the third indicator being indicative of the balance between the neuronal excitation and neuronal inhibition.

11. An apparatus according to claim 10, wherein the indicator unit is configured to display the third indicator on a continuous scale.

12. An apparatus according to claim 8, wherein
the first calculation unit is configured (i) to decompose the brain wave signal data into a first subband indicative of the neuronal excitation, to obtain first output data representing a time series of a quantitative characteristic of the brain wave signal data on the first subband and (ii) to determine the first indicator as one of a first measure indicative of the entropy of the first output data and a second measure indicative of a normalized form of k:th order central moment of the first output data; and
the second calculation unit is configured (i) to decompose the brain wave signal data into a second subband indicative of the neuronal inhibition, to obtain second output data representing a time series of a quantitative characteristic of the brain wave signal data on the second subband and (ii) to determine the second indicator as one of a third measure indicative of the entropy of the second output data and a fourth measure indicative of a normalized form of k:th order central moment of the second output data,
wherein k is an integer higher than three.

13. An apparatus according to claim 12, wherein the first indicator is indicative of the entropy of the first output data and the second indicator is indicative of the entropy of the second output data, wherein the first and second output data comprise wavelet coefficients.

14. An apparatus according to claim 13, wherein the first subband is substantially in its entirety above 16 Hz and the second subband is substantially in its entirety below 16 Hz.

15. An apparatus according to claim 8, further comprising a measurement unit configured to obtain brain wave signal data from a subject.

16. A method for the selection of an anti-epileptic drug for a subject with epileptiform activity, the method comprising:
deriving a first indicator indicative of the level of neuronal excitation;
deriving a second indicator indicative of the level of neuronal inhibition; and
selecting, based on the first and second indicators, an anti-epileptic drug for the subject.

17. A method according to claim 16, further comprising providing information on the selected anti-epileptic drug to a clinician.

18. A method according to claim 16, further comprising administering the selected anti-epileptic drug to the subject.

19. An apparatus for monitoring epileptiform activity, the apparatus comprising:
first calculation means for deriving a first indicator from brain wave signal data obtained from a subject, the first indicator being indicative of the level of neuronal excitation;
second calculation means for deriving a second indicator from the brain wave signal data, the second indicator being indicative of the level of neuronal inhibition; and
indicator means for giving, based on the first and second indicators, an indication of the level of at least one of the neuronal excitation and neuronal inhibition.

20. A computer readable medium comprising program code adapted to carry out, when run on a computer, the steps of:
deriving a first indicator from brain wave signal data obtained from a subject, the first indicator being indicative of the level of neuronal excitation;
deriving a second indicator from the brain wave signal data, the second indicator being indicative of the level of neuronal inhibition; and
giving, based on the first and second indicators, an indication of the level of at least one of the neuronal excitation and neuronal inhibition.
